(19) United States
(12) Patent Application Publication
(10) Pub. No.: US 2023/0346294 A1
(43) Pub. Date: Nov. 2, 2023

(54) AUXILIARY DETERMINATION DEVICE FOR EVALUATING EFFICACY OF TRANSCRANIAL MAGNETIC STIMULATION FOR PATIENT WITH DEPRESSION

(71) Applicants: Cheng-Ta Li, Taipei (TW); Chung-Ping Chen, Taipel (TW)

(72) Inventor: Shou-Hong Hung, Kaohsiung (TW)

(21) Appl. No.: 18/026,347

(22) PCT Filed: Sep. 15, 2020
(86) PCT No.: PCT/CN2020/115179

§ 371 Date: Mar. 14, 2023

(51) Int. Cl. A61B 5/372; A61B 5/00; A61B 5/16

(52) U.S. Cl. CPC A61B 5/372; A61B 5/7282; A61B 5/7264; A61B 5/725; A61B 5/165

(57) ABSTRACT

An auxiliary determination device for evaluating whether a transcranial magnetic stimulation (TMS) is effective for a patient with depression is provided. The device includes a feature extraction unit and a machine learning unit electrically connected thereto. In an interpretation mode, the feature extraction unit extracts a feature value from electroencephalography signals of the patient, and at least a classifier of the machine learning unit determines the efficacy of TMS for the patient according to the feature value of the electroencephalography signals. The electroencephalography signals are electroencephalography signals of the patient after being driven by a cognitive operation or a difference between electroencephalography signals before and after being driven by the cognitive operation, and the feature value is a linear or non-linear feature value. The auxiliary determination device of the invention can pre-evaluate the efficacy of TMS for the patient for avoiding ineffective treatment and unnecessary medical expense.

### FIELD OF TECHNOLOGY

The invention relates to an auxiliary determination device for assisting a doctor in evaluating a treatment for a patient with depression, and more particularly to an auxiliary determination device for evaluating an efficacy of a transcranial magnetic stimulation (TMS) for the patient with depression and a method for interpretating parameters for a transcranial magnetic stimulator.

### BACKGROUND

Depression might be triggered by endocrine abnormalities in human body, psychological stress or psychological trauma caused from major events. With the fast pace of life and high pressure of work for modern people, the proportion of patients with depression is gradually increased. Depression might cause inconveniences to patients in daily life, work, study and sleep, and major depressive disorder (MDD) even could be a serious mental disorder for patients. In addition to causing the patients become disable in daily life, work, study and sleep, about 60% of suicides are caused by major depressive disorder.

For patients with depression, especially those with major depressive disorder, it is necessary to have treatment for preventing regrets from happening. Current treatments for depression include medication, psychological counseling and transcranial magnetic stimulation. The medication may be oral or given by injection. The transcranial magnetic stimulation may be repetitive transcranial magnetic stimulation (r-TMS) or intermittent theta burst stimulation (i-TBS). The transcranial magnetic stimulator for performing the transcranial magnetic stimulation has many parameters that may be set, wherein after a portion of particular parameters of the transcranial magnetic stimulator are adjusted to particular values, the above-mentioned repetitive transcranial magnetic stimulation or intermittent theta burst stimulation may be generated.

Compared with drugs or the psychological counseling, the transcranial magnetic stimulation is a more expensive treatment, but the treatment period thereof for improving the syndrome of patients with depression is significantly shorter than that of drugs and the psychological counseling. However, unfortunately, the treatment of transcranial magnetic stimulation is not effective for every patient with depression, so the usage of transcranial magnetic stimulation to treat depression is still not popular. Moreover, due to the high cost, the patients with depression are mostly unwilling to try the treatment of transcranial magnetic stimulation.

### SUMMARY

Based on at least one of the above-mentioned objects, the invention provides an auxiliary determination device for evaluating whether a transcranial magnetic stimulation is effective for a patient with depression. The device comprises a feature extraction unit and a machine learning unit electrically connected to the feature extraction unit. In an interpretation mode, the feature extraction unit extracts at least one feature value from electroencephalography signals of the patient, and at least one classifier of the machine learning unit determines the efficacy of the transcranial magnetic stimulation for the patient according to the at least one feature value of the electroencephalography signals. The electroencephalography signals are electroencephalography signals of the patient after being driven by a cognitive operation or a difference between electroencephalography signals before and after being driven by the cognitive operation, and the at least one feature value is a linear or non-linear feature value.

Moreover, the auxiliary determination device further comprises a signal pre-processing unit electrically connected to the feature extraction unit for performing a signal pre-processing on the electroencephalography signals in the interpretation mode, wherein the signal pre-processing comprises at least one of a bandpass filtering, a resampling, and an independent component analysis.

Moreover, the auxiliary determination device further comprises a frequency band screening unit electrically connected to the feature extraction unit and the signal pre-processing unit for screening frequency bands of the electroencephalography signals in the interpretation mode to acquire the electroencephalography signals within particular frequency bands for subsequent feature extraction and signal interpretation.

Moreover, the particular frequency bands are α, β, γ, θ and δ frequency bands.

Moreover, the auxiliary determination device further comprises an electroencephalography signal measuring unit electrically connected to or communicated with the signal pre-processing unit for measuring the electroencephalography signals.

Moreover, the electroencephalography signals are acquired through at least one electrode of Fp1, Fp2, F3, F4, F7, F8, and Fz of the electroencephalography signal measuring unit.

Moreover, the at least one feature value comprises at least one of a largest Lyapunov exponent, an approximate entropy, a correlation dimension, a fractal dimension, a detrended fluctuation, a band power of fast Fourier transform, and a band power of Welch periodogram.

Moreover, the at least one classifier is a classifier based on a support vector machine, an adaptive boost, or a neural network architecture.

Moreover, the at least one classifier is a plurality of classifiers, and each of the plurality of classifiers is corresponding to a set of parameters of a transcranial magnetic stimulator.

Moreover, the parameters of the transcranial magnetic stimulator comprise mode, frequency, burst period, burst duration, rest interval, signal strength, and pulse number of each burst.

Based on at least one of the above-mentioned objects, the invention further provides a method for deciding parameters of a transcranial magnetic stimulator. The method comprises following steps. In an interpretation mode, through the feature extraction unit, at least one feature value from the electroencephalography signals of the patient is extracted, wherein the electroencephalography signals are electroencephalography signals of the patient after being driven by a cognitive operation or a difference between electroencephalography signals before and after being driven by the cognitive operation, and the at least one feature value is a linear or non-linear feature value, and then, through a plurality of classifiers of the machine learning unit, the efficacy of the transcranial magnetic stimulation for the patient is determined according to the at least one feature value of the electroencephalography signals, wherein each classifier is corresponding to one set of parameters for the transcranial magnetic stimulator.

The invention is advantageous that:

The auxiliary determination device and the method for deciding parameters for the transcranial magnetic stimulator provided in the invention can pre-evaluate that if the transcranial magnetic stimulation is effective to the patient for avoiding the ineffective treatment and unnecessary medical expense.

### BRIEF DESCRIPTION OF THE DRAWINGS

FIG. 1 is a functional block diagram of an auxiliary determination device for evaluating whether a transcranial magnetic stimulation is effective for a patient with depression according to a first embodiment of the invention.

FIG. 2 is a functional block diagram of an auxiliary determination device for evaluating whether a transcranial magnetic stimulation is effective for a patient with depression according to a second embodiment of the invention.

FIG. 3 is a schematic view showing an arrangement of a plurality of electrodes of an electroencephalography signal measuring unit on human's scalp according to an embodiment of the invention.

FIG. 4 is a flow chart of a method for deciding parameters of a transcranial magnetic stimulator in a training mode according to an embodiment of the invention.

FIG. 5 is a flow chart of a method for deciding parameters of a transcranial magnetic stimulator in an interpretation mode according to an embodiment of the invention.

### DESCRIPTION OF AN EMBODIMENTS

In order to further illustrate the objects, technical features and effects, please refer to the following detailed description about embodiments of the invention and the attached drawings to understand embodiments of the invention in detail.

An embodiment of the invention provides an auxiliary determination device for evaluating whether a transcranial magnetic stimulation is effective for a patient with depression and a method for deciding parameters for a transcranial magnetic stimulator. The concept is as follows. The transcranial magnetic stimulation utilizes magnetic waves to stimulate and change action potentials of nerve cells in brains of some patients with depression, thereby changing the activity of the stimulated brain region and thus improving syndromes of the patients with depression. Therefore, in an embodiment of the invention, the auxiliary determination device and the method for deciding parameters are based on at least one feature value extracted from electroencephalography signals of the patient with depression after receiving and being driven by a cognitive operation (e.g., programmed rostral anterior cingulate cortex (r-ACC)-engaging cognitive task (RECT) or transcranial magnetic stimulation, but not limited thereto), and at least one classifier which is already trained based on a machine learning training is utilized to assist in a determination whether the transcranial magnetic stimulation is effective for the patient with depression and decide the parameters for the transcranial magnetic stimulator according to the extracted feature value. Accordingly, the auxiliary determination device and the method for deciding parameters in an embodiment of the invention are capable of assisting the doctor in evaluating in advance if the transcranial magnetic stimulation is suitable for treating the patient with depression and also deciding the parameters for the transcranial magnetic stimulator to avoid the ineffective treatment and unnecessary medical expense.

Moreover, since electroencephalography signals are complex, non-linear and non-stationary signals, it is difficult to purely employ a linear extraction to extract the feature value thereof for expressing dynamic variations of complex neural activities. Accordingly, in an embodiment of the invention, in addition to express the features of the electroencephalography signals in time domain and frequency domain through a signal transformation (e.g., wavelet transform, but not limited thereto), a non-linear extraction and a linear extraction are also used to extract the feature value for further expressing the dynamic variations of the complex neural activities, so as to achieve an auxiliary determination for the efficacy of the transcranial magnetic stimulation for the patient with depression and also decide how to adjust the parameters of the transcranial magnetic stimulator for effectively treating the patient with depression according to the feature value.

In an embodiment of the invention, the feature value extracted through the non-linear extraction is, for example, a largest Lyapunov exponent (LLE), an approximate entropy, a correlation dimension, a fractal dimension, and a detrended fluctuation, but not limited thereto. The feature value extracted through the linear extraction is, for example, a band power of fast Fourier transform or Welch periodogram, but not limited thereto. That is, the feature value is a linear or non-linear feature value. Preferably, in an embodiment of the invention, more than two feature values are extracted, and more than two feature values comprise linear or non-linear feature values.

Furthermore, for improving the accuracy of the auxiliary determination and the parameters decision, in an embodiment of the invention, the electroencephalography signals are further processed, such as through a bandpass filtering and/or an independent component analysis (ICA), for eliminating noises from the electroencephalography signals. And, for further reducing the processing time, in an embodiment of the invention, a re-sampling of a down-sampling is performed on the electroencephalography signals. In conclusion, the auxiliary determination device and the method for deciding the parameters provided in an embodiment of the invention are easily implemented with short processing time, so an auxiliary determination result may be provided automatically to the doctor in real time for evaluating if the transcranial magnetic stimulation is effective to the patient with depression, and the decided parameters for the transcranial magnetic stimulator also may be provided to the doctor for avoiding the ineffective treatment and unnecessary medical expense. Accordingly, the invention is capable of helping the patient with depression (even the patient with major depression disorder) who has a great response to the transcranial magnetic stimulation to receive the treatment of transcranial magnetic stimulation for rapidly relieving the syndrome, thereby reducing the inconvenience and regret caused by the disease.

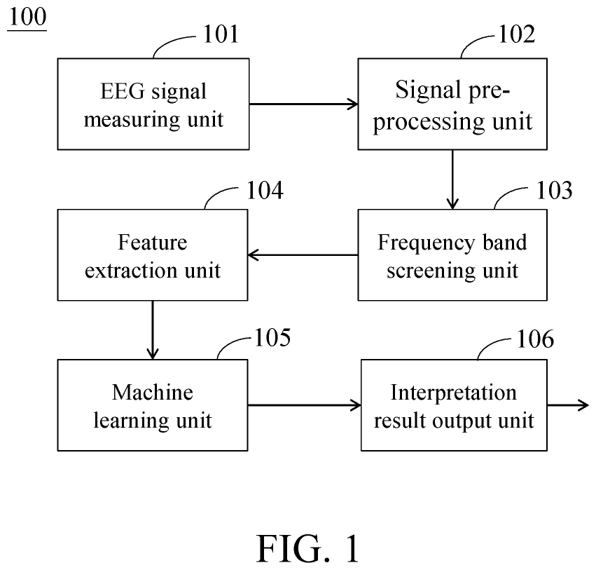

Following, please refer to FIG. 1. FIG. 1 is a functional block diagram of an auxiliary determination device for evaluating whether a transcranial magnetic stimulation is effective for a patient with depression according to a first embodiment of the invention. An auxiliary determination device 100 is a local apparatus located in a hospital or clinic center. The auxiliary determination device 100 comprises an electroencephalography (EEG) signal acquisition unit 101, a signal pre-processing unit 102, a frequency band screening unit 103, a feature extraction unit 104, a machine learning unit 105 and an interpretation result output unit 106. The electroencephalography signal measuring unit 101 is electrically connected to the signal pre-processing unit 102, the signal pre-processing unit 102 is electrically connected to the frequency band screening unit 103, the frequency band screening unit 103 is electrically connected to the feature extraction unit 104, the feature extraction unit 104 is electrically connected to the machine learning unit 105, and the machine learning unit 105 is electrically connected to the interpretation result output unit 106.

The electroencephalography signal measuring unit 101 may be a dry or wet electroencephalography signal measuring device with dry or wet electrodes. The quantity of electrodes may be 32, 64 or 128, and the invention is not limited by the type of the electroencephalography signal measuring device. Through the electroencephalography signal measuring unit 101, the electroencephalography signals of the patient after being driven by the cognitive operation may be acquired. In an embodiment of the invention, the efficacy of the transcranial magnetic stimulation for the patients with depression may be directly evaluated in accordance with the electroencephalography signals of the patient after being driven by the cognitive operation, or the efficacy of the transcranial magnetic stimulation for the patients with depression also may be evaluated in accordance with a difference between the electroencephalography signals before and after being driven by the cognitive operation. In this manner, the electroencephalography signal measuring unit 101 has to acquire electroencephalography signals of the patient before being driven by the cognitive operation.

The signal pre-processing unit 102 is utilized to pre-process the electroencephalography signals (namely, the electroencephalography signals of the patient after being driven by the cognitive operation or the difference between electroencephalography signals before and after being driven by the cognitive operation) transmitted from the electroencephalography signal measuring unit 101. The signal frequency of the electroencephalography signals is approximately at 60 Hz or less, so the signal frequency of the electroencephalography signals acquired by the electroencephalography signal measuring unit 101 is also approximately at 60 Hz or less. According to sampling theorem, the signals acquired by the electroencephalography signal measuring unit 101 are down-sampled at a sampling frequency which is more than twice the signal frequency, so as to avoid an aliasing distortion during reconstruction and also effectively reduce the amount of data and computations.

As described above, the signal frequency of the electroencephalography signals acquired by the electroencephalography signal measuring unit 101 is also approximately at 60 Hz or less, so it is possible to filter out the noises excluding the frequency band of 1-60 Hz through a bandpass filtering, e.g., a 1-60 Hz bandpass filtering. Furthermore, the above mentioned 1-60 Hz bandpass filtering also may be replaced by a low pass filtering for 60 Hz or less. The independent component analysis is used for finding independent components constructing the electroencephalography signals acquired by the electroencephalography signal measuring unit 101. Because when acquiring the electroencephalography signals, the minor movements of the mouth, nose, and eyes of the patient might influence the electroencephalography signals, through the independent component analysis, the independent components (comprising the noise components caused by the minor movements of the mouth, nose, and eyes of the patient, as well as the components constructing the electroencephalography signals) of the electroencephalography signals acquired by the electroencephalography signal measuring unit 101 may be found, and according thereto, the components of noises may be filtered out. That is, one of the purposes of signal pre-processing like the bandpass filtering and the independent component analysis is to filter out the noises. Here, the signal pre-processing unit 102 might not be necessary for the auxiliary determination device 100 and might be removed.

The frequency band screening unit 103 is used to screen the electroencephalography signals (namely, the electroencephalography signals of the patient after being driven by the cognitive operation or the difference between electroencephalography signals before and after being driven by the cognitive operation) transmitted from the electroencephalography signal measuring unit 101. Generally, electroencephalography signals are divided into five frequency bands comprising α (8-14 Hz), β (12.5-28 Hz), γ (25-60 Hz), θ (4-7 Hz) and δ (0.1-3 Hz) (the rare frequency bands are omitted here), so it is possible to screen the electroencephalography signals transmitted from the electroencephalography signal measuring unit 101 to acquire electroencephalography signals within a particular frequency band for the subsequent feature extraction and signal interpretation. For example, in the invention, it can determine if the repetitive transcranial magnetic stimulation is effective to the patient through only acquiring electroencephalography signals within θ frequency band to interpret, and alternatively, in the invention, it can determine if the intermittent theta burst stimulation is effective to the patient through only acquiring electroencephalography signals within β frequency band to interpret.

The frequency band screening unit 103 uses various transform methods for transforming the spatial domain or time domain signals into frequency domain signals to transform the electroencephalography signals into frequency domain signals so as to acquire electroencephalography signals within a particular frequency band. In an embodiment of the invention, preferably, the transform method may be wavelet transform, so as to simultaneously express the features in time domain and in frequency domain, but the invention is not limited thereto. Notably, in other embodiments, the interpretation also may be performed for all frequency bands of the electroencephalography signals, so the frequency band screening unit 103 might not be necessary and might be removed.

The feature extraction unit 104 extracts the feature value of the electroencephalography signals through the linear extraction and/or the non-linear extraction. The feature value extracted through the non-linear extraction is, for example, a largest Lyapunov exponent, an approximate entropy, a correlation dimension, a fractal dimension, and a detrended fluctuation, but not limited thereto. The feature value extracted through the linear extraction is, for example, a band power of Welch periodogram, but not limited thereto. The largest Lyapunov exponent indicates the instability or unpredictability of electroencephalography signals, and the detrended fluctuation represents the correlation between signals in remote time domain, so the feature value like the largest Lyapunov exponent and the detrended fluctuation actually represents the trend of electroencephalography signals. Other feature values for representing the trend of electroencephalography signals also may be extracted in the invention. The correlation dimension represents the influence degree of the signal value of electroencephalography signals at a current time point on the signal value at other time points, and the fractal dimension is used to quantify the degree of autocorrelation of electroencephalography signals, so the feature value like the correlation dimension and the fractal dimension actually represents the dimension of electroencephalography signals. Other feature values for representing the dimension of electroencephalography signals also may be extracted in the invention. The approximate entropy represents the regularity and complexity of electroencephalography signals, so the feature value like the approximate entropy actually represents the complexity of electroencephalography signals. Other feature values for representing the complexity of electroencephalography signals also may be extracted in the invention.

The machine learning unit 105 may comprise at least one classifier based on a support vector machine (SVM), an adaptive boost (Adaboost) or a neural network (NN) architecture, but the invention is not limited thereto. The classifier of the machine learning unit 105 is completed through learning and training, and the trained classifier classifies the electroencephalography signals according to the at least one feature value thereof for obtaining an interpretation result. The interpretation result is provided to the doctor through the interpretation result output unit 106. The interpretation result output unit 106 may be any kind of output apparatus, for example, a display, a communication unit, or a printer, and the invention is not limited thereby.

The machine learning unit 105 comprises a training mode and an interpretation mode. In the training mode, multiple sets of electroencephalography signals for training the classifiers are inputted into the machine learning unit 105 in sequence for learning. The multiple sets of electroencephalography signals for training the classifiers are electroencephalography signals corresponding to different sets of particular parameters which are effective for the transcranial magnetic stimulation, so through the training mode, it is able to obtain the trained classifiers for the efficacies of transcranial magnetic stimulations with different sets of particular parameters, for example, the classifier for the efficacy of the repetitive transcranial magnetic stimulation, the classifier for the efficacy of the intermittent theta burst stimulation, and the classifier for the efficacy of a sham (namely, a treatment for providing placebo effect). In the interpretation mode, according to the at least one feature value of the electroencephalography signals, a plurality of classifiers of the machine learning unit 105 can determine if the transcranial magnetic stimulation is effective to the patient and how to adjust the parameters of the transcranial magnetic stimulator. For example, when the classifier for the efficacy of the repetitive transcranial magnetic stimulation obtains an effective interpretation and the classifier for the efficacy of the intermittent theta burst stimulation obtains an ineffective interpretation, the interpretation result is indicated as effective, and the parameters of the transcranial magnetic stimulator should be set to perform the repetitive transcranial magnetic stimulation.

Without loss of generality, the parameters of the transcranial magnetic stimulator comprise mode, frequency, burst period, burst duration, rest interval, signal strength, and pulse number of each burst. The mode may be selected from the repetitive transcranial magnetic stimulation, the intermittent theta burst stimulation, a single and paired pulse transcranial magnetic stimulation (sp-TMS), an intermediate theta burst stimulation (im-TBS), a continuous theta burst stimulation (c-TBS) or a manual mode. The frequency is a frequency between pulses. The burst period is a period between two adjacent bursts. The burst duration is a duration of multiple continuous bursts. The rest interval is a rest interval after the multiple continuous bursts. The signal strength is a signal strength of each pulse. The pulse number of each burst is the number of pulse in one burst.

Through the trained classifiers for different sets of parameters and inputting the at least one feature value of the electroencephalography signals into each classifier, it is able to know which type of transcranial magnetic stimulation is effective to the patient, and also to decide the parameters of the transcranial magnetic stimulator, namely, the interpretation result comprises not only the information about the efficacy of the transcranial magnetic stimulation for the patient, but also the parameters for the transcranial magnetic stimulator.

Furthermore, when the machine learning unit 105 determines that there are more than two types of transcranial magnetic stimulations are effective to the patient through the trained classifiers, according to the interpretation result, the doctor can decide to use more than two types of transcranial magnetic stimulations to apply a cocktail treatment to the patient or to select one set of parameters for the transcranial magnetic stimulation to treat the patient. For example, when the interpretation result of the machine learning unit 105 indicates that both the intermediate theta burst stimulation and the single and paired pulse transcranial magnetic stimulation are effective to the patient, the doctor might decide to use one of which to treat the patient, or to treat the patient with the intermediate theta burst stimulation first and then treat the patient with the single and paired pulse transcranial magnetic stimulation.

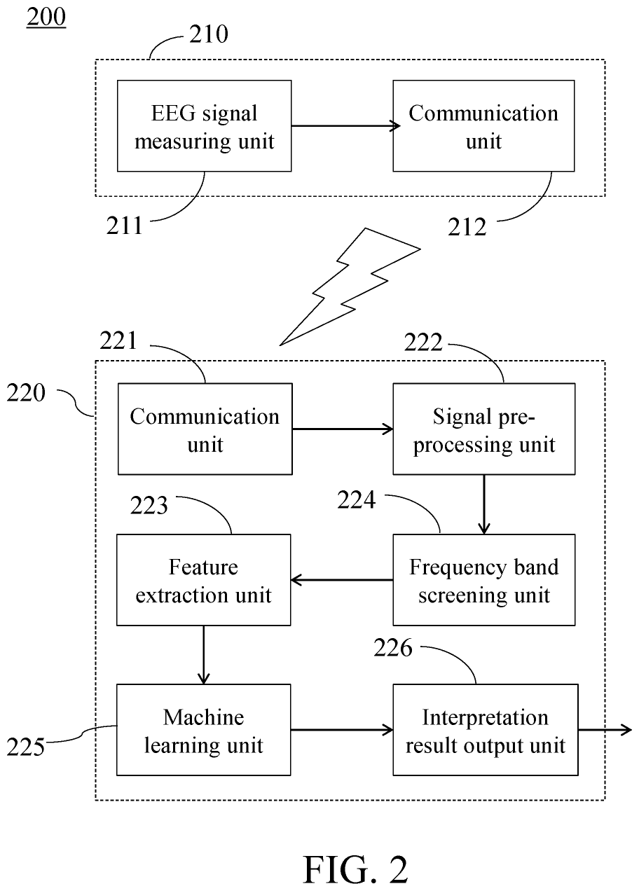

Please refer to FIG. 2. FIG. 2 is a functional block diagram of an auxiliary determination device for evaluating whether a transcranial magnetic stimulation is effective for a patient with depression according to a second embodiment of the invention. In this second embodiment, an auxiliary determination device 200 consists of an electroencephalography signal measuring apparatus 210 and a platform server 220 which are located at different locations, wherein the electroencephalography signal measuring apparatus 210 is located in a hospital or clinic center, and the platform server 220 may be located in a remote server center.

The electroencephalography signal measuring apparatus 210 comprises an electroencephalography signal measuring unit 211 and a communication unit 212, wherein the electroencephalography signal measuring unit 211 is electrically connected to the communication unit 212. The platform server 220 is configured into multiple functional blocks through its hardware and software codes, and comprises a communication unit 221, a signal pre-processing unit 222, a frequency band screening unit 223, a feature extraction unit 224, a machine learning unit 225, and an interpretation result output unit 226. The communication unit 221 is communicated with the communication unit 212 and is signally connected to the signal pre-processing unit 222. The signal pre-processing unit 222 is signally connected to the frequency band screening unit 223. The frequency band screening unit 223 is signally connected to the feature extraction unit 224. The feature extraction unit 224 is signally connected to the machine learning unit 225. The machine learning unit 225 is signally connected to the interpretation result output unit 226.

The electroencephalography signal measuring unit 211, the signal pre-processing unit 222, the frequency band screening unit 223, the feature extraction unit 224, the machine learning unit 225, and the interpretation result output unit 226 are identical to the electroencephalography signal measuring unit 101, the signal pre-processing unit 102, the frequency band screening unit 103, the feature extraction unit 104, the machine learning unit 105, and the interpretation result output unit 106 as shown in FIG. 1. The communication unit 212 is used for transmitting the electroencephalography signals measured by the electroencephalography signal measuring unit 211 to the communication unit 221, and the communication unit 221 transmits the received electroencephalography signals to the signal pre-processing unit 222.

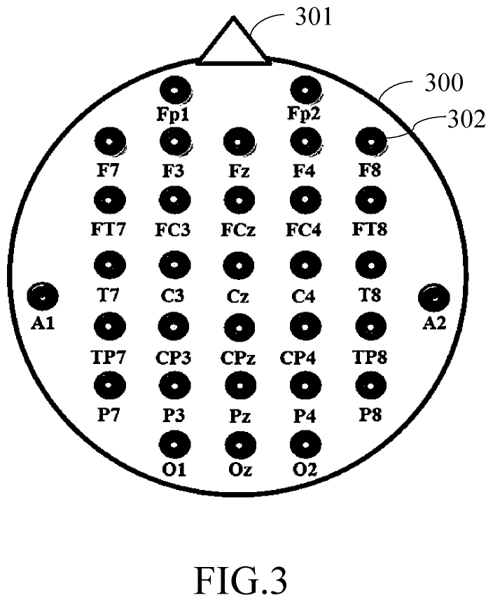

FIG. 3 is a schematic view showing an arrangement of a plurality of electrodes of the electroencephalography signal measuring unit on human's scalp according to an embodiment of the invention. In this embodiment, there are 32 electrodes 302 which are respectively indicated as A1, A2, Fp1, Fp2, F3, F4, F7, F8, Fz, FT7, FT8, FC3, FC4, FCz, T7, T8, C3, C4, Cz, TP7, TP8, CP3, CP4, CPz, P7, P8, P3, P4, Pz, O1, O2, and Oz, and the arranging positions thereof on human's scalp are shown in FIG. 3. In FIG. 3, the nose 301 of human is indicated to identify the relative front-rear and left-right positions of a human brain 300. These 32 electrodes 302 are identical to the 32 electrodes currently used in the common electroencephalography signal measuring unit, and the detailed description thereof is omitted. In the invention, preferably, the electroencephalography signals measured through only at least one electrode of Fp1, Fp2, F3, F4, F7, F8, and Fz is used to determine the efficacy of the transcranial magnetic stimulation for the patient.

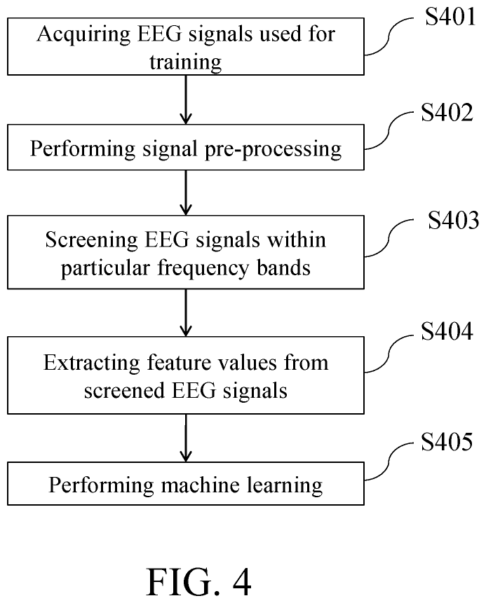

Please refer to FIG. 4. As described above, it is necessary to train each of the classifiers of the machine learning unit 105 in advance, so FIG. 4 shows a flow chart of a method for deciding parameters of the transcranial magnetic stimulator in a training mode according to an embodiment of the invention. First, in Step S401, the electroencephalography signals used for training are acquired, wherein the electroencephalography signals for training are electroencephalography signals of the patient after being driven by the cognitive operation or differential electroencephalography signals before and after being driven by the cognitive operation, and it is known that the electroencephalography signals for training are effective or ineffective to a particular set of parameters for the transcranial magnetic stimulation. Then, in Step S402, the signal pre-processing is performed on the electroencephalography signals for training, wherein the signal pre-processing is as described above and thus omitted here. Following, in Step S403, the frequency band screening is performed on the electroencephalography signals for training, wherein the frequency band screening is as described above and thus omitted here. In step S404, the feature extraction is performed on the electroencephalography signals for training, wherein the feature extraction is as described above and thus omitted here. In Step S405, the feature value of the electroencephalography signals for training is inputted into each of the classifiers for training the classifiers, and since it is known that the electroencephalography signals for training are effective or ineffective to a particular set of parameters for the transcranial magnetic stimulation, each classifier may be trained through multiple iterations.

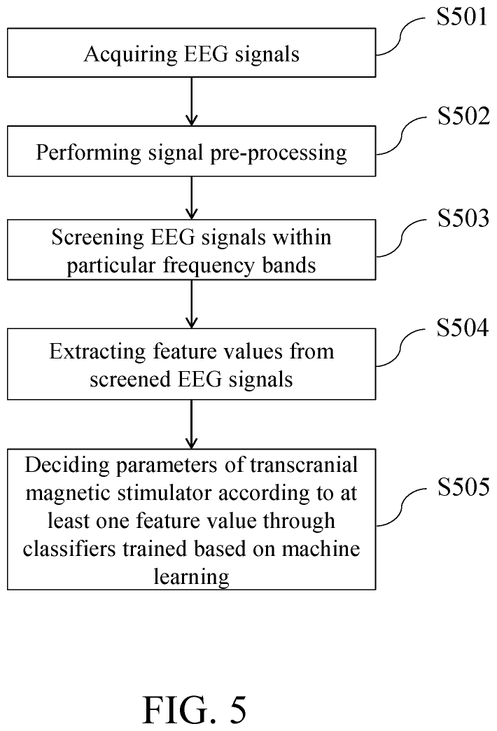

Furthermore, please refer to FIG. 5. FIG. 5 is a flow chart of a method for deciding parameters of the transcranial magnetic stimulator in the interpretation mode according to an embodiment of the invention. After the training for each classifier is completed, the electroencephalography signals are interpreted, so the doctor can decide which set of parameters of the transcranial magnetic stimulation is effective for treating the patient according to the interpretation result. First, in Step S501, the electroencephalography signals to be determined are acquired, wherein the electroencephalography signals are electroencephalography signals of the patient after being driven by the cognitive operation or differential electroencephalography signals before and after being driven by the cognitive operation, and it is still unknown that the electroencephalography signals to be determined are effective or ineffective to a particular set of parameters for the transcranial magnetic stimulation. Then, in Step S502, the signal pre-processing is performed on the electroencephalography signals to be determined, wherein the signal pre-processing is as described above and thus omitted here. Following, in Step 503, the frequency band screening is performed on the electroencephalography signals to be determined, wherein the frequency band screening is as described above and thus omitted here. In step S504, the feature extraction is performed on the electroencephalography signals to be determined, wherein the feature extraction is as described above and thus omitted here. In Step S505, the feature value of the electroencephalography signals to be determined is inputted into each of the classifiers for generating the interpretation result for the doctor to decide which set of parameters of the transcranial magnetic stimulation is effective for treating the patient.

In conclusion, as compared with the prior arts, the auxiliary determination device and the method for deciding parameters for the transcranial magnetic stimulator provided in embodiments of the invention at least comprise the following advantageous technical effects:

(1) The efficacy of the transcranial magnetic stimulation for the patient may be pre-evaluated for avoiding the ineffective treatment and unnecessary medical expense.

(2) There are many combinations of parameter sets for transcranial magnetic stimulators. The doctor can decide the particular set of parameters for the transcranial magnetic stimulation according to the interpretation result, so as to achieve an accurate treatment.

(3) The computations used in the auxiliary determination device and the method for deciding parameters for the transcranial magnetic stimulator are not complex and may be implemented easily.

The above description is only the detailed description and the drawings of some embodiments of the invention. However, features of the invention are not limited thereto and should not be used to limit the scope of the invention. All the scope of the invention shall be subject to the following claims. Any embodiment that is within the scope of the patent application of the invention or similar variations should be comprised in the scope of the invention. Any changes or modifications that may be easily considered by person skilled in the art of the invention may all be comprised in the patent scope of the invention.

What is claimed is:

1. An auxiliary determination device for evaluating whether a transcranial magnetic stimulation is effective for a patient with depression, characterized in that the auxiliary determination device comprising:
a feature extraction unit for extracting at least one feature value from electroencephalography signals of the patient in an interpretation mode, wherein the electroencephalography signals are electroencephalography signals of the patient after being driven by a cognitive operation or a difference between the electroencephalography signals before and after being driven by the cognitive operation, and the at least one feature value is a linear or non-linear feature value; and
a machine learning unit electrically connected to the feature extraction unit and comprising at least one classifier for determining whether the transcranial magnetic stimulation is effective for the patient according to the at least one feature value of the electroencephalography signals in the interpretation mode.

2. The auxiliary determination device of claim 1, characterized in that, further comprising:
a signal pre-processing unit electrically connected to the feature extraction unit for performing a signal pre-processing on the electroencephalography signals in the interpretation mode, wherein the signal pre-processing comprises at least one of a bandpass filtering, a resampling, and an independent component analysis.

3. The auxiliary determination device of claim 2, characterized in that, further comprising:
a frequency band screening unit electrically connected to the feature extraction unit and the signal pre-processing unit for screening frequency bands of the electroencephalography signals in the interpretation mode to acquire the electroencephalography signals within particular frequency bands for subsequent feature extraction and signal interpretation.

4. The auxiliary determination device of claim 3, characterized in that, wherein the particular frequency bands are α, β, γ, θ and δ frequency bands.

5. The auxiliary determination device of claim 2, characterized in that, further comprising:
an electroencephalography signal measuring unit electrically connected to or communicated with the signal pre-processing unit for measuring the electroencephalography signals.

6. The auxiliary determination device of claim 5, characterized in that, wherein the electroencephalography signals are acquired through at least one electrode of Fp1, Fp2, F3, F4, F7, F8, and Fz of the electroencephalography signal measuring unit.

7. The auxiliary determination device of claim 1, characterized in that, wherein the at least one feature value comprises at least one of a largest Lyapunov exponent, an approximate entropy, a correlation dimension, a fractal dimension, a detrended fluctuation, a band power of fast Fourier transform, and a band power of Welch periodogram.

8. The auxiliary determination device of claim 1, characterized in that, wherein the at least one classifier is a classifier based on a support vector machine, an adaptive boost, or a neural network architecture.

9. The auxiliary determination device of claim 1, characterized in that, wherein the at least one classifier is a plurality of classifiers, and each of the plurality of classifiers is corresponding to a set of parameters of a transcranial magnetic stimulator.

10. The auxiliary determination device of claim 9, characterized in that, wherein the parameters of the transcranial magnetic stimulator comprise mode, frequency, burst period, burst duration, rest interval, signal strength, and pulse number of each burst.
